(19) United States
(12) Patent Application Publication
(10) Pub. No.: US 2012/0296201 A1
(43) Pub. Date: Nov. 22, 2012

(54) Lesion Detection and Localization Using Gamma Cameras with Converging Collimation

(75) Inventor: Chuanyong Bai, Poway, CA (US)

(73) Assignee: DIGIRAD CORPORATION, Poway, CA (US)

(21) Appl. No.: 13/455,272

(22) Filed: Apr. 25, 2012

Related U.S. Application Data
(60) Provisional application No. 61/478,802, filed on Apr. 25, 2011.

(51) Int. Cl. A61B 6/00; A61B 10/02

(52) U.S. Cl. 600/424

(57) ABSTRACT

First and second gamma radiation detector heads are oriented to image an area of a subject. The area of said subject is completely within a field of view that is defined between the first and second gamma radiation heads. Focal points of each of the first and second gamma radiation heads are also within an area defined between the first and second gamma radiation heads. A computer is programmed to receive image information from both the first gamma radiation detector head and the second gamma radiation detector head, and operating to use information from both the first gamma radiation detector head and the second gamma radiation detector head, as well as to use information indicative of a distance between the first gamma radiation detector head and the second gamma radiation detector head, to determine a location of an item of interest in the subject and between the first gamma radiation detector head and the second gamma radiation detector head, by calculating using information about similar triangles formed from known positions of the first gamma radiation detector head and the second gamma radiation detector head, and the information.

This application claims priority from provisional application No. 61/478,802, filed Apr. 25, 2011, the entire contents of which are herewith Incorporated by reference.

### BACKGROUND

Breast cancer detection using anatomical imaging modalities, such as CT, X-ray, MRI, etc suffers from a lack of functional information. Cancerous tissue may not be differentiated from dense breast tissues in these modalities. Gamma cameras can be used to identify cancerous tissues that are of higher radiopharmaceutical uptake than health tissues. For example, a dual-head gamma camera has been developed by Gamma Medica, Inc. for breast cancer detection.

When suspicious cancerous tissues or tissues with increased uptake are detected, biopsy may be needed to confirm if the tissues are truly cancerous. Consequently, a localization mechanism is needed to guide the biopsy procedure.

A gamma camera that can be used for both the detection and localization of cancerous tissues is therefore highly desirable. US patent publication 20100016865 A1, published Jan. 21, 2010, describes an approach to use a planar gamma camera for this purpose. The camera has two segments with slant-hole collimation to obtain two images of the objects in the common field of view (FOV). The location of the object of interest can be derived from the two images.

The current inventors, however, have noticed drawbacks of the approach in 20100016865 as follows. The transaxial field-of-view (in the planes parallel to the detector surface) is determined by the slant angle and the distance from the transaxial plane to the detector surface. The FOV is proportional to the distance from the transaxial plane to the detector surface and the tangent of the slant angle. Because of this reason, the FOV for planes close to the detector surface is very small. This requires the detector or the patient have to be translated during the imaging, increasing the imaging time and the workload for imaging;

Also, the resolution of the collimation in 20100016865is poorer for objects farther away from the detector plane. This resolution decrease further decreases the FOV.

The acquisition time/image quality compromise in 20100016865 is poor due to the detector/patient translation that is made necessary to overcome the small FOV.

The resolution of the localization in the transaxial plane is determined by the collimator resolution in 20100016865. Consequently, the localization resolution decreases for objects farther away from the detector surface;

The resolution of the localization in the direction perpendicular to detector surface is 0.5/tan(theta) times that of the resolution in the transaxial plane, where theta is the slant angle. For theta=20°, 0.5/tan(theta)=1.37. This means, the resolution of localization is 37% worse in the perpendicular direction than the transaxial direction in 20100016865.

When applied to procedures such as imaging guided biopsy, the technique in 20100016865, therefore, suffers from the small FOV, long acquisition time for imaging (therefore long total procedure time), high noise level due to the time allowed for each position of the detector relative to the object, and poor localization resolution.

A technique developed by Ashburn in U.S. Pat. No. 6, 055,450 uses a bifurcated gamma camera system for lesion detection and imaging guided biopsy. The two heads are connected using a hinge-like mechanism and a gap is defined so that a medical device such as the biopsy device can be accommodated.

The hinge-like mechanism allows the two heads to form different angles from 0 to 180 degrees, so the imaging FOV, distance from the heads to the object of interest (thus resolution), can be adjusted for optimal procedure.

The technique in Ashburn can have various FOVs at different configurations. The transaxial FOV (common space imaged by both the heads in a plane that is in between the two heads along the half-angle direction between the two heads) is nearly the same as the dimension of the heads when the angle between the heads is 0 degree, and reduces to its minimum when the angle is 180 degrees.

However, the axial FOV (common space imaged by both the heads in the direction perpendicular to the heads) is minimal (in fact 0) when the angle between the heads is 0 degree (because there is no space between the two heads when the two heads are fully folded) and maximal when the angle between the two heads is 180 degrees.

The major issues in Asburn include that

(1) Because of the gap between the two heads, the FOV is farther away from the detector surfaces, therefore, the localization resolution is decreased;

(2) The transaxial FOV and axial FOV tradeoff. When a relatively large axial FOV is needed, the angle between the two heads needs to be large; at 180 degrees, the system performs similar to that in [2] with slightly larger transaxial FOV but the localization resolution is poorer because the average distance of the FOV is farther away from the detector heads due to the gap. Note that we use two slant-hole collimator for the analysis here;

(3) Even though the angle between the two heads can be varied, there is a range of the angle in which the axial localization is none or very poor. Assume the two heads have slant collimator with slant angles Θ1 and Θ2, then when the two heads are at angle Θ1+Θ2, the axial resolution (or depth information of lesions) will be lost because the slant holes of the two heads are aligned at this angle. At angles Θ near this value, the axial resolution is very poor. The angle between the heads that leads to the highest axial resolution is θ1+θ2+90°.

(4) The technique does not provide a unified three-dimension FOV for imaging and biopsy, thus, does not allow precise procedure, such as robot controlled biopsy.

In another U.S. Pat. No. 5,961,457, Raylman and Wahl describe radiopharmaceutical-guided biopsy. One or multiple gamma camera heads acquire images at multiple angles (greater than or equal to 2). Operators then choose a first and second view to display and locate a lesion in the two views. The computer then calculate the lesion center in the views and using a sinogram calculation to convert the lesion location in view images to Cartesian coordinates to guide the biopsy.

Drawbacks of U.S. Pat. No. 5,961,457 include that:

(1) Even though the inventors mention that only two views are needed to locate a lesion, the invention essentially acquires multiple views of data and requires the user to choose two out of the multiple views to identify and localize the lesions. The rest of the views are in general not used. Therefore, similar to the situation in [2], the two views used for lesion localization only account for a portion of the total acquisition time. Users have to either increase the overall imaging time to obtain enough counts or compromise the image quality to not increase the overall imaging time.

(2) Because multiple views are acquired, real time imaging guiding for biopsy is not practical, meaning, the biopsy has to be done after the image acquisition.

(3) Detectors may need to rotate around the object, or a tunnel shaped detector (such as using PET) is needed. This will limit the arrangement of the biopsy apparatus.

(4) Even though the inventors mention that only two views are necessary to locate a lesion, they did not describe how to acquire the data if only two views can be acquired and if and how the apparatus should be different than when multiple views are acquired, not alone to say optimization.

(5) The invention required the image of a fiducial marker to determine the position of a lesion to the known fiducial makers in the Cartesian coordinate.

(6) Users are required to identify lesions in the two views they choose, a software means is than used to calculate the centroid (location) of the lesion in the views, then translate into scanner coordinates and finally translated into Cartesian coordinates for biopsy. Depending upon the angle between the two views, the resulted localization resolution will suffer from the similar situation as described above.

### SUMMARY

Embodiments describe a gamma camera system for functional imaging as well as lesion localization with large field of view (“FOV”) and improved resolution. With large FOV, no movement of the detector relative to the object is required, therefore allowing longer imaging time, better image quality, and single coordinate system for both imaging FOV and biopsy guidance. With improved resolution, the functional image quality can be improved and the accuracy (resolution) of localization is also improved.

### BRIEF DESCRIPTION OF THE DRAWINGS

In the Drawings:

FIG. 1 shows a block diagram of the system according to the embodiments.

### DETAILED DESCRIPTION

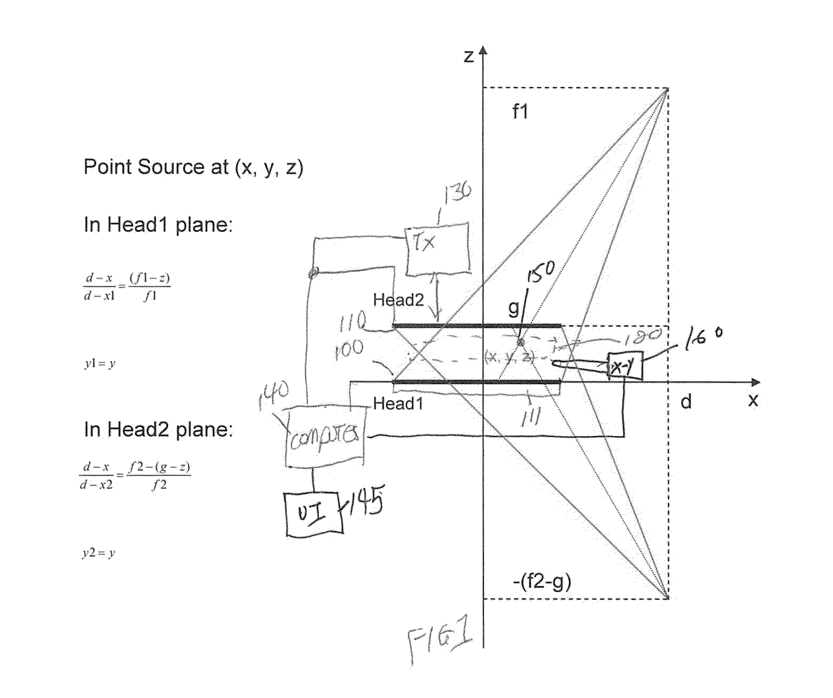

An embodiment is shown in FIG. 1. FIG. 1 shows a gamma camera system which has a first gamma camera head 100 and a second gamma camera head 110. The two heads are planar detectors that are facing one another. The object to be imaged 120 is located between the two heads. In the embodiment, head 1 is fixed, but head 2 can be moved by a translation device 130 to vary the distance g between the heads 1 and 2. The detectors can use a fan beam collimator such as 111 outside of the transaxial plane. This means that the entire area between the heads defines the field of view of these heads.

The output signal from the heads is fed to a computer 140 which carries out the functions described herein.

At least one of the two detectors 100, 110 uses converging beam collimation (fan-beam or cone-beam) collimation. The imaging FOV is determined by the system geometry. Image resolution and localization resolution does not decrease with the distance from the detectors.

The two detectors 100 and 110 acquire two planar images of the object. These planar images are processed by software running in the processor 140. The processor simultaneously generates a 3D image from the two planar images. This drives a biopsy device, which can be a core biopsy using a needle operating from sides of the imaging system so the image acquisition can acquire data during the biopsy procedure. This provides a real time imaging guided biopsy with improved image quality because of effectively longer acquisition time.

The user identified lesions to be biopsied in the 3D image together with the linked two planar images and 3-view images for improved lesion localization.

Advantages of the embodiment include:

1. Large FOV;

2. No need for camera and/or patient translation during imaging;

3. A 3D volume images is generated simultaneously during the image acquisition and biopsy, together with the two planar image obtained, lesion localization accuracy can be improved;

4. Improved image resolution (due to the use of converging collimation; consequently more accurate functional images;

5. Improved localization resolution, more accurate biopsy guidance;

6. Single coordinate system for both imaging and biopsy guidance; thus easier and more accurate.

FIG. 1 depicts one embodiment with two detectors positioned face to face and the object (such as a human breast) in between the two detectors. Each detector is equipped with a fan-beam collimator. When projected to the transaxial plane, the focal-point of the fan-beam collimator is out of the area corresponding to the detectors and the focal-points of the two collimators project to the same point in the transaxial plane. For another embodiment, the geometry can be that the focal-points of the two collimators project to different positions in the transaxial plane, depending upon the desired application.

Based on the geometry shown in FIG. 1, the system can operate as follows to localize a position of the lesion. A point source (a lesion 150) in the FOV is imaged by both of the detectors 110, 120. The relationship of the detected positions of the point source in the two detectors and the location of the point source in the FOV is shown in the following equations:

Point Source at (x, y, z)

In Head1 plane:

$$\frac{d - x}{d - x 1} = \frac{( f 1 - z )}{f 1}$$

$$y 1 = y$$

In Head2 plane:

$$\frac{d - x}{d - x 2} = \frac{f 2 - ( g - z )}{f 2}$$

$$y 2 = y$$

### (1) Localization

From the relationship between the location of the point source in the object and the imaged positions in the detector planes of Head 1 and Head 2 shown in FIG. 2, we can derive the location of the point source in the imaging FOV from the imaged positions as follows when using fan-beam geometry:

$$\begin{matrix}x = \frac{\begin{matrix}( f 1 - g ) \cdot d \cdot x 1 + ( f 2 - g ) \cdot d \cdot x 2 - \\ ( f 1 + f 2 - g ) \cdot x 1 \cdot x 2 + g \cdot {d}^{2}\end{matrix}}{f 1 \cdot ( d - x 2 ) + f 2 \cdot ( d - x 1 )} & ( 1 ) \\ y = y 1 = y 2 & ( 2 ) \\ z = \frac{f 1 \cdot f 2 \cdot ( x 2 - x 1 ) + f 1 \cdot g \cdot ( d - x 2 )}{f 1 \cdot ( d - x 2 ) + f 2 \cdot ( d - x 1 )} & ( 3 )\end{matrix}$$

When using two fan-beam collimators with the same focal-length, i.e., f1=f2=f, then equations (1) and (3) can be rewritten as:

$$\begin{matrix}x = \frac{( f - g ) \cdot d \cdot ( x 1 + x 2 ) - ( 2 f - g ) \cdot x 1 \cdot x 2 + g \cdot {d}^{2}}{f \cdot ( 2 d - x 1 - x 2 )} & ( 4 ) \\ z = \frac{f \cdot ( x 2 - x 1 ) + g \cdot ( d - x 2 )}{2 d - x 1 - x 2} & ( 5 )\end{matrix}$$

If Head 1 is parallel (f1>>f2), then equations (1) and (3) become:

$$\begin{matrix}x = x 1 & ( 6 ) \\ z = g - \frac{x 1 - x 2}{d - x 2} \cdot f 2 & ( 7 )\end{matrix}$$

### (2) FOV

Assume the detector dimension in x (the fan-direction) is 2 L, the FOV in x direction is (assume f1=f2=f):

$$\begin{matrix}\begin{matrix}FOV ( x ) = ( d + L ) \cdot \frac{f - g / 2}{f} - ( d - L ) \cdot \frac{f}{f - g} \\ = ( \frac{f - g / 2}{f} - \frac{f}{f - g} ) d + ( \frac{f - g / 2}{f} + \frac{f}{f - g} ) L\end{matrix} & ( 8 ) \\ If f >> g , then FOV ( x ) = 2 L - ( 1.5 d / f ) \cdot g . & ( 9 )\end{matrix}$$

Localization Resolution

Using a converging-beam collimator, the resolution in the transaxial plane improves with the increased distance from the detector surface due to the amplification effect in the converging direction.

Equations (5) and (8) can be used to obtain the best compromise of z-resolution of localization and FOV. From equation (5), the resolution in the z direction is about f/d times that of the resolution in the transaxial direction. If choosing d=f/2, then the resolution in z direction is about the same as the transaxial direction. This eliminates the anisotropic resolution issue in [2] and [3].

Image Quality Improvement

Since the depth information (location of the lesions in z direction) can be obtained, such information can further be used to improve the image of the lesions using deconvolution techniques as described in C. Bai, R. Conwell R, “An iterative deconvolution technique for planar scintigraphic imaging,” (abstract) J. Nucl. Med. 47, 2006.

Using deconvolution techniques, image resolution can be significantly improved. The improved resolution can further improve the localization resolution of the lesions and the definition of the size and shape of the lesion for a more accurate biopsy.

If the heads use pixelated gamma cameras, then an oversampling approach such as in C. Bai, R. Conwell R, H. Babla, J. Kindem, “Improving image resolution using oversampling for pixelated solid-state Gamma cameras,” J. Nucl. Med. 52, 2011.can be used to decrease the sampling pixel size by a factor of two, and consequently, the intrinsic resolution of imaging as well as localization can be improved by a factor of two. Note that using this approach, one or multiple small movements of the heads relative to the object are needed, such as 1.6 mm translation in the detector plane. For example, when using 4 samplings, one can image the object for about ¼ of the projected imaging time at one head position, then translate the head to the next position followed by image for about ¼ of the projected imaging time and so on.

The two detectors do not need to be facing each other in one embodiment. Head2 can be operated at an angle theta relative to Head 1 if some space is required to improve biopsy procedure.

Head 1 and Head 2 can be equipped with different collimators, but at least one should use a converging collimation. Note that from equation (7), one can see that the localization resolution in z direction is about f/2d times that in the transaxial plane when one head is parallel and the other is fan-beam. The resolution is poorer than when both of the two heads use fan-beam collimation (f/2d times that in the transaxial plane).

Both single photon and positron emitters can be used according to embodiments. When positron emitters are used, high energy collimators should be used for sufficient collimation of the photons.

A mechanical system such as 160 can be integrated with the camera shown in FIG. 1 for accurate control of the equipment used for biopsy. The mechanical system will use the same coordinate system as the one used for imaging FOV. Use of such a system can hence easily and accurately guide the biopsy procedure.

The following describes the overall system and its operation according to an embodiment.

Two planar detectors 100, 110 face each other, each having the same dimension, with offset fan-beam collimation illustrated in FIG. 1. A translation device 130 is controlled by a controlling computer to move Head 2 up and down (farther and closer) to Head 1. The computer also measures information, such as the x direction, for identification of FOV in x direction for biopsy control.

The computer runs software that automatically generates and displays a 3D image of the imaging FOV and the FOV in the x direction based on the distance from Head 2 to Head 1. This information is used to position the object in the FOV for imaging and biopsy.

The computer generates coordinates of the FOV for both imaging and biopsy.

The computer acquires the emission image from the object in the FOV;

The computer automatically and simultaneously generates a 3D volume image from the planar image acquired on Head 1 and Head 2 by backprojecting the each of the two planar images into the imaging FOV followed by summation of the two backprojected images.

The computer can then automatically display the two planar images and the 3D volume images, as well as a 3-view image of the 3D image.

The computer can optionally run a routine to improve planar image quality as well as localization using deconvolution techniques, followed by, repeating steps above.

The computer 140 includes a user interface 145 that provides a means of controlling the biopsy device 160 to point to and locate a lesion from the 3D-view images as well as the two planar images and the 3D image. All the views are cross-referenced so that a cross-hair will be put on the same lesion in all the images to improve the localization accuracy and confidence of the user. A 3D (x, y, z) location of the lesion in the coordinates of the imaging and biopsy FOV is then generated and displayed on the screen.

According to another embodiment, information can be used by computing a center of mass of the lesion identified by the user on the user interface, compute the lesion location in the two planar images then use equations (1) to (3) to calculate the 3D (x, y, z) location of the lesion in the biopsy FOV.

In another embodiment, the two approaches of localization can be used together to improve localization confidence also;

Once the user has identified the areas of the lesions for biopsy, these areas are stored in the computer. The biopsy device 160 then automatically uses the lesion locations identified above for automated biopsy.

All of the above can be done as software on computers.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, this can be used with the other kinds of medical imaging.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments of the invention.

The various illustrative logical blocks, modules, and circuits described in connection with the embodiments disclosed herein, may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, display port, or any other form.

When operated on a computer, the computer may include a processor that operates to accept user commands, execute instructions and produce output based on those instructions. The processor is preferably connected to a communication bus. The communication bus may include a data channel for facilitating information transfer between storage and other peripheral components of the computer system. The communication bus further may provide a set of signals used for communication with the processor, including a data bus, address bus, and/or control bus.

The communication bus may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or any old or new standard promulgated by the Institute of Electrical and Electronics Engineers (“IEEE”) including IEEE 488 general-purpose interface bus (“GPIB”), and the like.

A computer system used according to the present application preferably includes a main memory and may also include a secondary memory. The main memory provides storage of instructions and data for programs executing on the processor. The main memory is typically semiconductor-based memory such as dynamic random access memory (“DRAM”) and/or static random access memory (“SRAM”). The secondary memory may optionally include a hard disk drive and/or a solid state memory and/or removable storage drive for example an external hard drive, thumb drive, a digital versatile disc (“DVD”) drive, etc.

At least one possible storage medium is preferably a computer readable medium having stored thereon computer executable code (i.e., software) and/or data thereon in a non-transitory form. The computer software or data stored on the removable storage medium is read into the computer system as electrical communication signals.

The computer system may also include a communication interface. The communication interface allows' software and data to be transferred between computer system and external devices (e.g. printers), networks, or information sources. For example, computer software or executable code may be transferred to the computer to allow the computer to carry out the functions and operations described herein. The computer system can be a network-connected server with a communication interface. The communication interface may be a wired network card, or a Wireless, e.g., Wifi network card.

Software and data transferred via the communication interface are generally in the form of electrical communication signals.

Computer executable code (i.e., computer programs or software) are stored in the memory and/or received via communication interface and executed as received. The code can be compiled code or interpreted code or website code, or any other kind of code.

A “computer readable medium” can be any media used to provide computer executable code (e.g., software and computer programs and website pages), e.g., hard drive, USB drive or other. The software, when executed by the processor, preferably causes the processor to perform the inventive features and functions previously described herein.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media. The computer readable media can be an article comprising a machine-readable non-transitory tangible medium embodying information indicative of instructions that when performed by one or more machines result in computer implemented operations comprising the actions described throughout this specification.

Operations as described herein can be carried out on or over a website. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

Also, the inventors intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The programs may be written in C, or Java, Brew or any other programming language. The programs may be resident on a storage medium, e.g., magnetic or optical, e.g. the computer hard drive, a removable disk or media such as a memory stick or SD media, or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A system, comprising:
a first gamma radiation detector head, oriented to image an area of a subject;
a second gamma radiation detector head, facing said first gamma radiation head, and also oriented to image said area of said subject;
where said area of said subject is completely within a field of view that is defined between said first and second gamma radiation heads, and focal points of each of said first and second gamma radiation heads are within an area defined between said first and second gamma radiation heads; and
a computer, receiving image information from both said first gamma radiation detector head and said second gamma radiation detector head, and operating to use information from both said first gamma radiation detector head and said second gamma radiation detector head, as well as to use information indicative of a distance between said first gamma radiation detector head and said second gamma radiation detector head, to determine a location of an item of interest in said subject and between said first gamma radiation detector head and said second gamma radiation detector head, by calculating using information about similar triangles formed from known positions of said first gamma radiation detector head and said second gamma radiation detector head, and said information.

2. The system as in claim 1, wherein said first gamma radiation detector head and said second gamma radiation detector head have the same size.

3. The system as in claim 2, wherein said first gamma radiation detector head and said second gamma radiation detector head each use offset fan beam collimation, and locations of said offset fan beam collimation form parts of said similar triangles.

4. The system as in claim 1, further comprising a translation device, operating to move one of the heads relative to the other of the heads, where a distance between the heads sets parts of the similar triangles.

5. The system as in claim 1, further comprising a biopsy controller, that uses said location of interest to guide a biopsy device toward said location of interest.

6. The system as in claim 1, wherein said computer automatically and simultaneously generates a 3D volume image from planar images from said heads by backprojecting each of the planar images into the field of view to form backprojected images, followed by summation of the backprojected images.

7. The system as in claim 1, wherein said computer improves planar image quality as well as localization using deconvolution techniques.

8. The system as in claim 5, wherein said computer computes a center of mass of the item of interest, and uses said center of mass to guide said biopsy device.

9. A method of medical imaging, comprising:
imaging an area of a subject with a first gamma radiation detector head and also with a second gamma radiation detector head, while maintaining said area of said subject completely within a field of view that is defined between said first and second gamma radiation heads, and focal points of each of said first and second gamma radiation heads are within an area defined between said first and second gamma radiation heads; and
receiving image information from both said first gamma radiation detector head and said second gamma radiation detector head into a computer that is programmed to use information from both said first gamma radiation detector head and said second gamma radiation detector head, as well as to use information indicative of a distance between said first gamma radiation detector head and said second gamma radiation detector head, to determine a location of an item of interest in said subject and between said first gamma radiation detector head and said second gamma radiation detector head, by calculating using information about similar triangles formed from known positions of said first gamma radiation detector head and said second gamma radiation detector head, and said information.

10. The method as in claim 9, wherein said first gamma radiation detector head and said second gamma radiation detector head have the same size.

11. The method as in claim 10, wherein said first gamma radiation detector head and said second gamma radiation detector head each use offset fan beam collimation, and locations of said offset fan beam collimation form parts of said similar triangles.

12. The method as in claim 9, further comprising using the computer to control moving one of the heads relative to the other of the heads, where a distance between the heads sets parts of the similar triangles.

13. The method as in claim 9, further comprising controlling a biopsy operation to use said location of interest to guide a biopsy device toward said location of interest.

14. The method as in claim 9, wherein said computer automatically and simultaneously generates a 3D volume image from planar images from said heads by backprojecting each of the planar images into the field of view to form backprojected images, followed by summation of the backprojected images.

15. The method as in claim 9, wherein said computer improves planar image quality as well as localization using deconvolution techniques.

16. The method as in claim 13, wherein said computer computes a center of mass of the item of interest, and uses said center of mass to guide said biopsy operation.
